(19) United States
(12) Patent Application Publication
(10) Pub. No.: US 2019/0335995 A1
(43) Pub. Date: Nov. 7, 2019

(54) METHODS AND APPARATUS FOR IN VIVO CHARACTERIZATION OF THE LENS CRYSTALLIN AGGREGATION INDEX

(71) Applicant: INTEGRATED FIBER OPTIC SYSTEMS (IFOSYS), INC., Setauket, NY (US)

(72) Inventor: Harbans S. Dhadwal, Setauket, NY (US)

(21) Appl. No.: 16/373,033

(22) Filed: Apr. 2, 2019

Related U.S. Application Data
(60) Provisional application No. 62/665,240, filed on May 1, 2018.

(51) Int. Cl. A61B 3/117; A61B 3/12; A61B 3/15; G01N 15/02

(52) U.S. Cl. CPC A61B 3/1173; A61B 3/1225; G01N 2015/0222; G01N 15/0211; A61B 3/152

(57) ABSTRACT

An ophthalmic diagnostic system, method and medium for determining the lens crystallin index includes: focused delivery of a coherent source to a measurement volume in the interior of the lens; a second focused delivery of a coherent source is combined with the first source for precise location of the measurement volume; a third coherent optical path collects the backward scattered signal from the random movement of crystallins within the lens; a power splitter obtains two copies of the backward scattered signal; cross/auto correlation of the backward scattered signal is performed and modulated over a time scale; and various time scale signatures are processed obtain the lens crystalline index. The system, method, and medium are self-correcting and account for abnormal counter values.

### CROSS-REFERENCE TO RELATED APPLICATION

This application claims the benefit of U.S. Provisional Patent Application No. 62/665,240, filed on May 1, 2018, the entire contents of which are incorporated herein by reference.

### BACKGROUND

### Technical Field

The present disclosure relates to in vivo, quantitative methods for characterization of the transparency of a living eye lens, and to methods and apparatus that can track localized changes in the lens transparency from the age of 25 to 55 years. More particularly, the present disclosure relates to integrated fiber optic based dynamic light scattering apparatus and methods including an electronic processing system for the characterization of the physical properties of the lens through a measurement of the cross/auto correlation function of the backscattered signal from a focused beam of coherent radiation in the lens.

### Background of Related Art

Generally, dynamic light scattering (DLS) aka photon correlation spectroscopy or quasi-elastic light scattering, refers to a sensitive method that provides for coherent illumination of an unknown system of scatterers in motion, and the coherent detection of the modulation resulting from the motion, and the subsequent analysis of the modulated signal, leading to the recovery of physical parameters characterizing the motion or the size, shape and speed of the scattering species.

U.S. Pat. No. 5,284,149 discloses the use of DLS for in vitro measurements from excised lenses from both animals and humans, but the non-imaging design of the backscatter fiber optic probe was not suitable for in vivo measurement of the human eye lens. U.S. Pat. No. 5,815,611 discloses an imaging backscatter fiber optic probe, which enables in vivo DLS measurements of the eye, see, “In vivo dynamic light scattering characterization of a human lens: cataract index,” Current Eye Research, vol. 20, No. 6, pp. 502-510 (2000). U.S. Pat. No. 5,973,779 discloses another imaging backscatter probe for in vivo DLS of the human eye. Ansari, R. R., et al., “Measuring Lens Opacity: Combining Quasi-Elastic Light Scattering with Scheimpflug Imaging System,” Proceedings of SPIE, 3246: 35-42 (1999). However, these ophthalmic systems have shortcomings in reliability and repeatability due to abnormalities arising from reflex and blinking by human subjects, as well as, obtaining a consistent measure of the progressing molecular changes taking place in the aging lens.

U.S. Pat. No. 5,706,072 discloses an ophthalmic measuring apparatus based on DLS. The apparatus includes an optical detecting system for detecting and excluding abnormalities in the scattered light intensity by means of photosensors and electronic shutters. However, the abnormality detection system is too slow to detect and exclude abnormalities in scattered light at the high-speed sampling times scales of 10 ns to 500 ns. The inherent latency in the detection and operation of the electronic shutters is of the order of milliseconds.

U.S. Pat. No. 7,236,250 discloses a DLS method based on phase-modulation and interference. Generally, phase-modulation methods, using homodyne detection, require substantially more components, as well as stabilized optical platforms for obtaining data of sufficiently high quality.

U.S. Pat. No. 8,467,067 discloses the use of DLS to extract the size of particles below 100 nm. The system is based on the use of low-coherence sources and path-length-resolved dynamic light scattering. While the technique has some utility in concentrated systems, however, it also requires a very stable optical platform to perform the heterodyne signal detection needed. These severe limitations make DLS methods based on phase-modulation unusable for in vivo ophthalmic applications.

U.S. Pat. No. 8,388,134 discloses a method and apparatus for performing quasi-elastic light scattering and fluorescent ligand scanning on a subject's eye. While also suffering from the above limitations the apparatus performs DLS at a scattering angle of 90°, restricting measurements to a central region of the lens, along the optical axis.

In addition to the technological limitations of the above methods, a reliable and quantifiable measure of the clarity (or lack of clarity) of the lens is still lacking.

### SUMMARY

The ophthalmic diagnostic systems and methods of this disclosure overcome the above short comings and provides a real-time measurement of the clarity of the lens through a single quantifiable parameter, Laser Crystallin Index (LCX), which shows measurable changes at least in the age group from 35 years to 55 years, see, Abazari, A, and Dhadwal H, “Utility of Vision Index Pen in detecting early cataract and loss of accommodation,” Abstract 6016 2018 ARVO Annual Meeting, Apr. 29, 2018, Honolulu, Hi. In addition to the technological advantages of the present disclosure, the apparatus and methods herein disclosed also decrease the costs of the ophthalmic device compared with prior systems, which use costly digital correlators.

It is an object of the present disclosure to provide an in vivo ophthalmic diagnostic apparatus for determining the lens crystallin aggregation index in the eye.

It is another object of the present disclosure to provide hardware for directly recovering the lens crystallin index from the auto/cross-correlation of the backward scattered laser light.

It is another object of the present disclosure to provide a hardware abnormal count sensor and discriminator.

It is another object of the present disclosure to provide a portable, relatively inexpensive apparatus for the lens crystallin index, and a fiber optic transreceiver for use in such an apparatus.

It is another object of the present disclosure to provide a hardware laser diode driver circuit breaker for added operational safety.

It is another object of the present disclosure to provide a field programmable gate array correlator and a math processor to have a low cost portable device.

A Vision Index Pen diagnostic system of the present disclosure, as well as other embodiments, objects, features and advantages of the present disclosure will be apparent from the following detailed description, which is to be read in connection with the accompanying drawings. It is to be understood, however, that the drawings are designed as an illustration only and not as a definition of the limits of this disclosure.

Provided in accordance with aspects of the present disclosure is a system for dynamic light scattering measurement provided in accordance with the present disclosure includes first and second light sources, first and second gradient index lenses, first and second optical paths from the first and second light sources to an eye by way of the first and second gradient index lenses, respectively, an optical splitter configured to split light, originating from the first and second light sources and scattered from the eye, into first and second light signals, first and second light detectors configured to detect the first and second light signals, respectively, and a correlator configured to correlate respective first and second signals output from the first and second light detectors.

In an aspect of the present disclosure, the first and second light sources are configured to produce different wavelengths of light.

In another aspect of the present disclosure, the correlator is a cross-correlator.

In yet another aspect of the present disclosure, the correlator is an FPGA autocorrelator.

In still another aspect of the present disclosure, the correlator includes an abnormal count sensor and discriminator configured to reject counter values falling outside a specified range to inhibit distortion of measurement.

In still yet another aspect of the present disclosure, first and second single-mode optical fibers are configured to define a portion of the first and second optical paths, respectively. In such aspects, the first and second single-mode optical fibers and the respective first and second gradient lenses may be incorporated into respective first and second transreceivers extending through a body of an eye probe.

In another aspect of the present disclosure, a circuit breaker is configured to shut down at least the first light source if emitted optical power from the first light source reaches a threshold.

A method for extracting a lens crystallin index and non-transitory computer readable medium having stored thereon instructions which, when executed by one or more processors, perform the method for extracting a lens crystallin index include obtaining measurements of backscattered light originating from a plurality of light sources and reflected by an eye, performing a data inversion of at least some of the obtained measurements to obtain inverted data, performing a fitting of at least some of the inverted data, and generating a lens crystallin index data plot based on a result of the fitting.

In an aspect of the present disclosure, obtaining measurements includes splitting the backscattered light into a plurality of light signals, detecting the plurality of light signals, and correlating the plurality of light signals to determine a counter value, wherein the obtained measurements are counter values.

In another aspect of the present disclosure, at least some of the obtained measurements are discarded and not used in the data inversion. In such aspects, obtained measurements within a counter value range are used in the data inversion and obtained measurements outside of the counter value range are not used in the data inversion.

In yet another aspect of the present disclosure, at least some of the inverted data is not used in the fitting. In such aspects, the inverted data at or below an upper limit is used in the fitting and the inverted data above the upper limit is not used in the fitting.

### BRIEF DESCRIPTION OF THE DRAWINGS

The above and other objects, features, and advantages of certain embodiments of the present invention will be more apparent from the following detailed description taken in conjunction with the accompanying drawings, in which:

FIG. 1 is a block diagram of the Vision Index Pen ophthalmic diagnostic system, according to embodiments of the present disclosure;

FIG. 2 is a schematic of an eye probe of the Vision Index Pen diagnostic system of FIG. 1, according to embodiments of the present disclosure;

FIG. 3 is a schematic of a fiber optic transreceiver of FIG. 2, according to embodiments of the present disclosure;

FIG. 4 is a schematic of a laser diode circuit breaker of the Vision Index Pen diagnostic system of FIG. 1, according to embodiments of the present disclosure;

FIG. 5 is a block diagram showing the instructional steps for operating the Vision Index Pen diagnostic system of FIG. 1, according to embodiments of the present disclosure;

FIG. 6 is an example of the interactive display of the Vision Index Pen diagnostic system of FIG. 1, according to embodiments of the present disclosure;

FIGS. 7(a)(1)-7(c)(1) and 7(a)(2)-7(c)(2) are plan and frontal views, respectively, illustrating a sequence of steps used for locating the measurement volume inside the lens during the alignment procedure of the Vision Index Pen diagnostic system of FIG. 1, according to embodiments of the present disclosure;

FIGS. 8(a) and 8(b) show the results obtained from an observational clinical study establishing efficacy of the Vision Index Pen diagnostic system of FIG. 1, according to embodiments of the present disclosure;

FIG. 9 is another of a Vision Index Pen ophthalmic diagnostic system, according to embodiments of the present disclosure;

FIG. 10 is a block diagram of a FPGA correlator of the Vision Index Pen diagnostic system of FIG. 9, according to embodiments of the present disclosure;

FIG. 11 is a block diagram of a FPGA math processor of the Vision Index Pen diagnostic system of FIG. 9, according to embodiments of the present disclosure;

FIGS. 12(a) and 12(b) show the abnormal counts arising from reflex and blinking by human subjects during measurement using the Vision Index Pen diagnostic system of FIG. 1, according to embodiments of the present disclosure; and

FIG. 13 is a block diagram of an abnormal count sensor and discriminator of the Vision Index Pen diagnostic system of FIG. 1, according to embodiments of the present disclosure.

### DETAILED DESCRIPTION

While the presently disclosed apparatus and methods for determining the lens crystallin aggregation index may have utility for characterizing the transparency of the human eye lens, may additionally or alternatively have utility in determining the state of aggregation of other systems containing small molecules, such as aggregation of proteins during the synthesis of large single crystal proteins. As another example of a utility of the presently disclosed apparatus and methods, the state of aggregation of small molecules in the formulation of ophthalmic drugs may be determined. Thus, without limiting the applicability of the presently disclosed apparatus and methods to determining the lens crystallin aggregation index of the eye lens, the present disclosure detailed below provides exemplary embodiments with regard to determining the lens crystallin index of the eye lens.

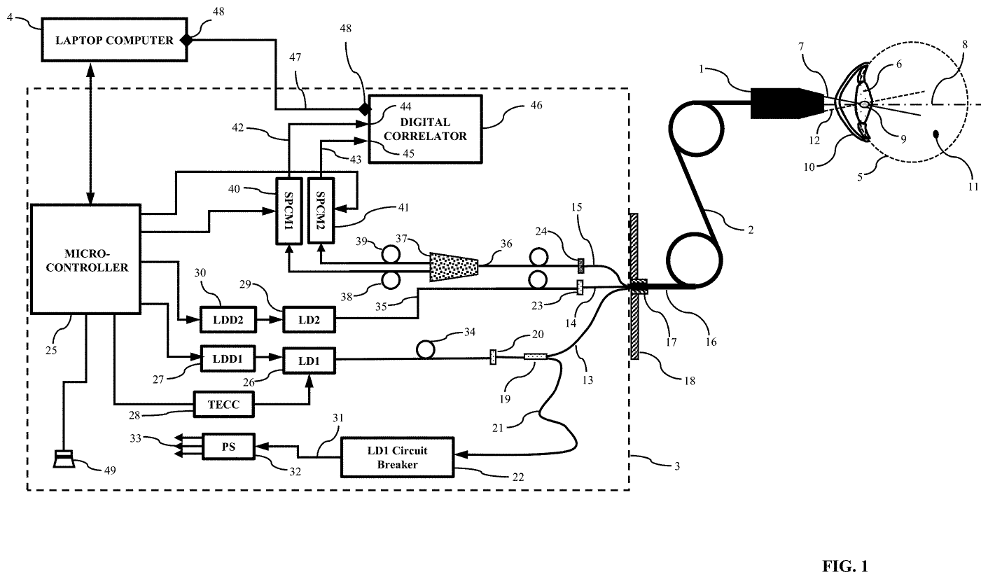

Referring to FIG. 1, there is shown, a representation of the various components of the Vision Index Pen diagnostic system (VIPDx) for determining the lens crystallin aggregation index (LCX). The LCX provides a quantitative measure of the lens crystallin aggregation index, which is an indicator of lens transparency and presbyopia. Typically, low values of LCX in the range of 20-30 are indication of 100% lens transparency and visual accommodation values greater than 8. LCX values in the range of 50 to 200 are early indicators of presbyopia, visual accommodation values in the range 3 to 8, and clinically undetectable changes in the loss of lens transparency. Values of LCX greater than 200 are indicative of clinically observable changes in lens transparency, showing formation of cataracts and visual accommodation values below 2. The LCX values are localized within a small measurement volume, typically a cylindrical volume, with a diameter of 20 μm to 60 μm and a length of 250 μm to 500 μm. The spatial distribution of the LCX estimates, from various parts of the lens from the nucleus to the cortical regions, can be used as a predictor of the aging effects on the optical properties of the lens. As such, the methods and apparatus of the present disclosure can define subject specific time intervention points at which the progressing ocular diseases, such as blindness and presbyopia, can be arrested by the administration of a therapeutic agent.

According to FIG. 1, the VIPDx system comprises of three sub-assemblies: 1) an eye probe head 1, which is connected to the electronic hardware module through a fiber optic tether cable 2; 2) an electronics module 3; and 3) a computer 4, e.g., a laptop computer, desktop computer, tablet, smart phone, etc., running software for both controlling the measurement sequence and for analyzing the measured data, in real time, in order to obtain, for example, the LCX value, and the variance in the LCX value. Other physical parameters describing the state of the lens 6, while not discussed here, can also be recovered from the data.

The eye probe 1 is positioned in front of the eye 5 as shown in FIG. 1; however, it should be noted that typically), the eye probe 1 may be mounted on a slit lamp or ophthalmoscope (not shown), which are a common visual examination apparatus utilized by opticians to examine various regions of the eye 5, including the lens 6. During the alignment phase, two focused coherent beams 7 and 8, emanate from the eye probe 1, having a common cross-over region defining a measurement volume 9. The measurement volume 9 can be position at any point from the anterior surface 10 of the cornea, to some point in the vitreous region 11 of the eye 5. The extent of penetration into the vitreous region is determined by the design of the eye probe 1. In the illustrated embodiment, the cross-over region is at 15 mm to 25 mm from the tip of the eye probe 1, allowing access to the vitreous region, beyond the posterior surface of the lens 6. A third coherent optical path 12 in the eye probe 1 is provided for collection of the scattered signal from the measurement volume 9, in the backward direction, typically, at an obtuse angle in the range 150° to 170° between the transmitting beam 7 and the collecting beam 12.

The tether cable 2 is a strengthened, flexible steel tube with a black PVC coating. It shrouds three single-mode optical fibers 13, 14, 15, of which single-mode optical fibers 13, 14 deliver coherent signal beams to the eye probe 1 and single-mode optical fiber 15 collects the back scattered signal from the measurement volume 9. The proximal end portion 16 of the tether is fixed into a bushing 17 which is fastened to the front panel 18 of the enclosure of the electronic module 3, which houses the electronics and other devices. The proximal end portion of each of the single-mode optical fibers 13, 14, 15, is terminated with a connector, such as, FC/PC fiber optic connector. The connectorized single-mode optical fiber 13 is connected to one output arm of a 1×2 single-mode fiber optic power splitter 19, with a power splitting ratio 99:1. The input arm of the said 1×2 fiber optic coupler is connected to a variable optical attenuator 20. The second output arm 21 of the said 1×2 fiber optic coupler is connected to the input of the LD1 circuit breaker 22. The second single-mode optical fiber 14 is connected to an in-line FC/PC adapter 23. The third single-mode optical fiber 15 is connected to an in-line FC/PC adapter 24.

Software running on computer 4 or similar device controls the functioning of the VIPox system and guides the operator to make real-time, in vivo, measurements of the LCX in the measurement volume 9 of the lens 6. A microcontroller 25 receives instructions from the computer 4 and sends and receives signals from the various electronic devices mounted inside the enclosure of the electronics module 3. The primary coherent source 26 is driven from signals received from the driver board 27 which receives control signals from the microcontroller 25. Furthermore, the primary coherent source 26 is operated at constant temperature by means of a closed-loop temperature controller circuit 28. Temperature controller maintains the output signal power and spectral emission within the specified design limits of power and frequency of emission. A secondary coherent source 29 is driven from signals received from the driver board 30 which, in turn, receives control signals from the microcontroller 25.

The primary coherent source 26 is operated at two discrete power levels: a low power mode is used during the alignment step and a high-power mode is used during measurement. Secondary control of the optical power levels entering the lens 6 via the variable optical attenuators 20 and 23 enables the coherent sources 26 and 29 to be driven aggressively, ensuring spectral purity and power stability of the said sources. Spectral purity is directly proportional to the coherence length of the source, which increases the self-beating efficiency of the scattered signal, producing a correlation function of high signal quality. Further, the variable optical attenuator 20 is adjusted to ensure that the coherent source power level entering the lens 6 remains below the threshold power level for non-specific risk to the human eye 5. Direct current control of the coherent source 26 to achieve the eye-safe power levels is not desirable as this necessitates operation of the coherent source 26 near lasing threshold, resulting in poor spectral purity of the source emission and a correlation function of poor quality.

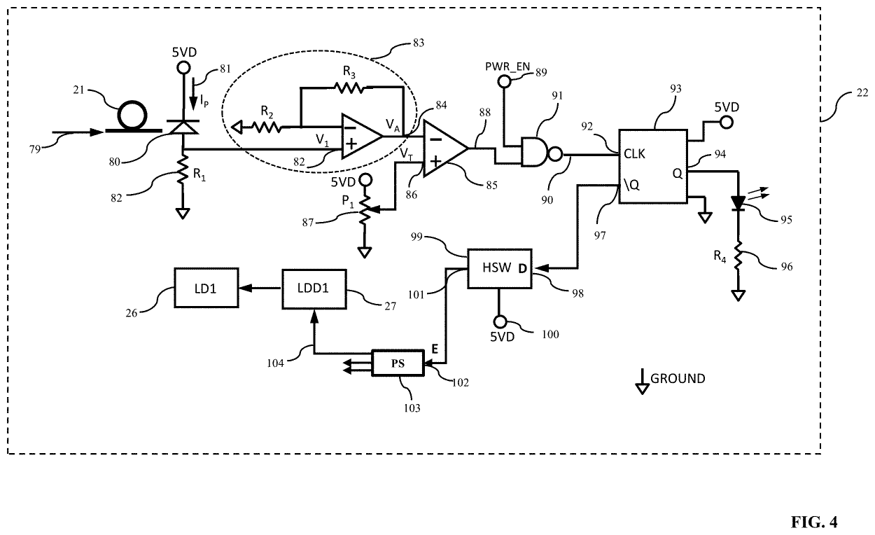

A further safety feature, LD1 circuit breaker 22, provides a control signal for immediate shut-down of the coherent source 26 if the emitted optical power level increases beyond the non-specific risk threshold. Here, circuit breaker 22 is implemented in hardware; however, software-based safety is also contemplated. The electronic circuit including circuit breaker 22 is designed to sense unpredictable increases in the optical power levels beyond a specified threshold and immediately turn off electrical power to the coherent source 26. The second output arm 21 of the 1×2 single-mode fiber optic power splitter 19, with a power splitting ratio of 99:1, collects 1% of the optical power entering the single-mode optical fiber 13. The LD1 circuit breaker 22, described below with reference to FIG. 4, senses the optical power level in the single-mode optical fiber 21, and generates a power shutdown control signal 31 when the sensed optical power passes the threshold level. The electrical power distributor circuit 32 immediately disables electrical power on the output bus 33 in response to the shutdown signal 31.

The secondary coherent source 29, driven by the driver 30 with signals issued from the microcontroller 25, is used during the alignment to visualize the measurement volume 9 in the interior of the lens 6 and as a fixation target during the data acquisition period. The output signals from the primary and secondary sources are transported through single-mode optical fibers 34 and 35, the in-line optical attenuators 20 and 24, and the tether cable 2, until they emerge from the distal tip of the eye probe 1, as two angled, converging beams 7 and 8, with a common-cross over region or measurement volume 9 inside the lens 6.

The backward scatterred signal from the lens 6 cytoplasm, within the measurement volume 9, is collected along the coherent optical path 12. The collected signal is transported by the single-mode fiber 15 in the tether 2 to the input arm 36 of a 1×2 single-mode optical power splitter 37, which divides the signal emanating from the single-mode fiber 15 into two equal portions which are coupled to two single-mode optical fibers 38 and 39. Single photon counting modules 40 and 41 convert the optical signal to voltage pulses 42 and 43 of equal height and width, but with varying frequency. Dependent on the strength of the optical signal, pulse frequency increases with the increasing signal power. The asynchronous voltage pulses 42 and 43 are coupled to two inputs 44 and 45, respectively, of a digital correlator 46. The digital correlator 46 is configured to measure the real time cross-correlation of the two voltage signals 42 and 43, or the autocorrelation of either one of the voltage signals 42 and 43. Cross-correlation cancels the dead-time effects of single photon counting modules 40 and 41, thereby extending the high-speed response of the system. The start/stop functions of the digital correlator 46 are issued from the control program running on the computer 4 via signal wire 47, terminated with RJ45 connectors 48 (or the like). A speaker 49 emits an audible beeping sound (or other suitable audible output) during the measurement cycle informing the patient to remain stationary, with minimal eye and head movement. When the measurement terminates, the audible beeping sound stops indicating that the patient can relax. Subsequently, the data from the digital correlator 46 is transferred to the computer 4 for real-time analysis and data processing to recover the LCX value.

During an eye examination, a fixation target is typically used to direct the attention of the patient in a desired direction, allowing the optician to access various components of the eye 5. In the VIPDx system, use of externally mounted fixation targets, as is common in commercial slit lamps, is not effective as such a target cannot be placed directly along the optical axis of the eye 5. In the embodiment of FIG. 1, the focused optical path 8, is along the optical axis of the eye 5 and serves as an alignment tool for the location of the cross-over region or measurement volume 9. The focused optical path 8 also serves as the fixation target for the patient during alignment and measurement. The second coherent source 29 has an emission wavelength different from that of the primary coherent source 26 so as not to corrupt the backscattered signal 12 from the measurement volume 9. The use of disparate emission wavelength for the two coherent sources 26 and 29 simplifies the alignment procedure as the cross-over of two spots of different color is easier to identify than the cross-over formed by spots of the same color. As an example, the coherent source 26 is operated in the single frequency mode with a nominal emission wavelength of 633 nm. The alignment/fixation coherent source 29 may have an emission wavelength of 488 or 532 nm. These wavelengths correspond to red, blue and green light spots, respectively. The reverse configuration is also contemplated.

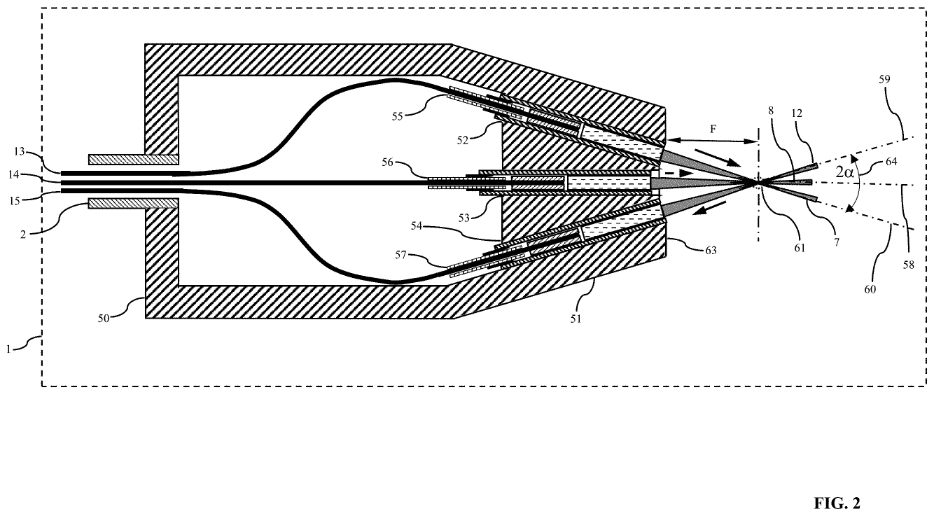

FIG. 2 illustrates the structure of the eye probe 1, which is typically fabricated with a black anodized aluminum body 50. However, it is understood that it could be another suitable color and/or constructed from any other suitable material, e.g., a hard plastic-material such as black Delrin®, available from E. I. du Pont de Nemours and Company. The tapered front end 51 is machined (or otherwise formed) with three precisely located cylindrical holes 52, 53, 54 to accept fiber optic transmitters 55, 56 and a fiber optic receiver 57, respectively. The fiber optic transmitters 55, 56 and receiver 57 are precisely positioned and fixed in the cylindrical holes such that the optical axis 58 of the fiber optic transmitter 56, the optical axis 59 of the fiber optic receiver 57 and the optical axis 60 of the fiber optic transmitter 55 have a common intersection point 61, which is located at a distance “F” from the front surface 63 of the eye probe. In embodiments such as illustrated in FIG. 2, “F” has a nominal value between 15 mm and 25 mm, and full inclination angle “2α” between the optical axes 59 and 60. A range of values for “2α” between 15° and 40° are typical for backscattered DLS systems.

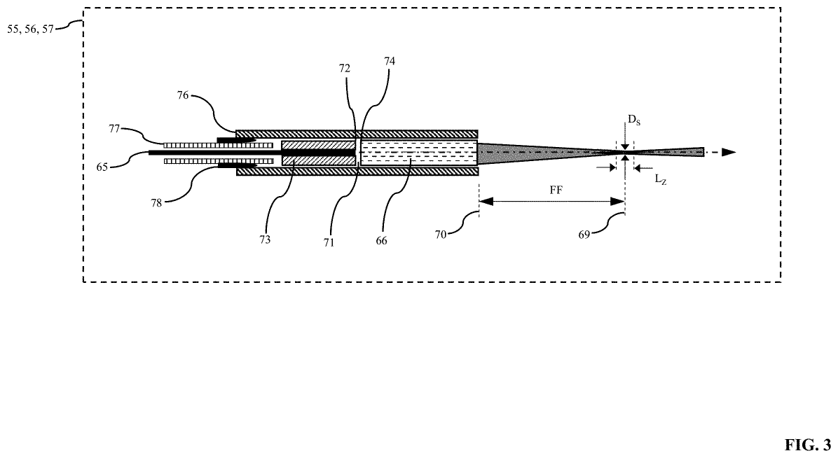

FIG. 3 illustrates the structure of fiber optic transmitters 55, 56, 57, aka as fiber optic transreceivers, used in the construction of the eye probe 1. A fiber optic transreceiver 55, 56, 57 is fabricated from a single-mode optical fiber 65, and a gradient index microlens 66. The fiber optic transreceiver 55, 56, 57 is designed based on the constraints imposed on the diameter “Ds” and the separation or image distance “FF”, between the intersection plane 69 and the distal end 70 of the gradient index microlens 66. Several combinations of a gradient microlenses 66 and a single-mode optical fiber 65 satisfy the design constraints. Design optimization may be an iterative process, leading to a transreceiver 55, 56, 57, comprising a particular gradient index microlens 66 and a single-mode fiber 65 to provide an appropriate length of the air gap 71 between the distal end 72 of the single-mode optical fiber 65, mounted in ferrule 73, and the proximal end 74 of the gradient index microlens 66. Typically, the image distance “FF,” is in the range of 15 mm to 25 mm and the diameter of the focused spot size “Ds” has a nominal range between 20 μm and 60 μm. The effective length “Lz” of the focused spot is twice the Rayleigh length. The gradient index microlens 66 and the mounted single-mode optical fiber 65 are fixed in a cylindrical tube 76, which may be made from stainless-steel. The single-mode optical fiber 65 is further encased in an opaque loose and flexible jacket 77, which may be black Teflon tubing. The jacket 77 is secured into the tube 76 by means of a suitable epoxy 78.

The VIPDx system uses a focused coherent source to interrogate the molecular composition of the eye lens 6. The device uses three safe-guards for safe operation below the exposure limit standards imposed by “ANSI Z80.36-2016 for Ophthalmics—Light Hazard Protection for Ophthalmic Instruments.” Referring back to FIG. 1, the first safe-guard uses a current-limiting resistor inside the laser diode driver 27 to limit the maximum output optical power emanating from the coherent source 26; the second safe-guard controls the output power of the coherent source 26 via the application software running on the computer 4.

FIG. 4 is a schematic of the electronic circuit breaker 22 of FIG. 1, which is the third safe-guard against unpredictable events that may cause a surge in the output power level of the coherent source 26. A small fraction 79 of the output power of the coherent source 26 is extracted by a pick-up optical fiber 21 (see also FIG. 1) and detected by a photodiode 80, which generates a current (I_{P}) 81. The resistor (R₁) 82 transform the current 81 (I_{P}) into a voltage (V₁) which is applied to the non-inverting input 82 of the non-inverting amplifier 83, which has a voltage gain (1+(R₃/R₂)). The amplified voltage (V_{A}) is applied to the inverting input 84 of the high-speed comparator 85. The voltage at the non-inverting input 86 of the comparator 85 is set by the potentiometer (P₁) 87 to correspond to the threshold optical power level for non-specific risk to the human eye 5 (FIG. 1).

During normal operation, the voltage (V_{A}) at the inverting input 84 of the comparator 85 is below the threshold voltage (V_{T}) at the non-inverting input 86. Under this condition the output 88 of the comparator is at HIGH voltage level. As such, the PWR_EN 89 is also HIGH such that the output state 90 of the NAND gate 91 is LOW. The clock input 92 of the JK flip-flop 93 is also LOW. The output Q 94 of the JK flip-flop 93 is low, thus the fault indicator LED 95 is OFF. The resistor (R₄) 96 limits the brightness of the LED 95. The complimentary output \Q 97 of the JK flip-flop 93 is HIGH. A HIGH level at the control input (D) 98 of the high side switch (HSW) 99 closes the switch, connecting voltage source (5VD) 100 to the output 101 of the high side switch 99, which is connected to the input (E) 102 of the power distribution block 103. One of the output power rails 104 supplies power to the laser diode driver 27 driving the primary coherent source driver 26 (see also FIG. 1).

The circuit breaker 22 is triggered if the voltage (V_{A}) at the inverting input 84 of the comparator 85 exceeds the threshold voltage (V_{T}) at the non-inverting input 86 of the comparator 85. When the voltage (V_{A}) exceeds the threshold voltage (V_{T}), the output of the comparator 88 goes from HIGH to LOW, causing the clock input 92 of the JK flip flop 93 to go from LOW to HIGH causing the output (Q) 94 to go HIGH, turning the fault LED 95 ON. The complimentary output /Q 97 goes LOW causing the high side switch 99 to OPEN. The power to the LDD1 driver 24 is immediately turned OFF, resulting in a rapid SHUTDOWN of the primary coherent source 26. Recovery from the triggered circuit breaker 22 requires a hardware restart.

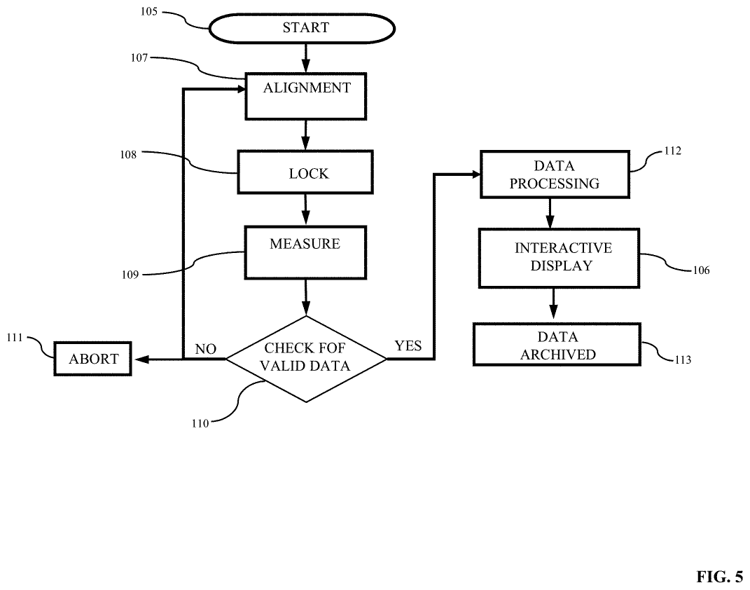

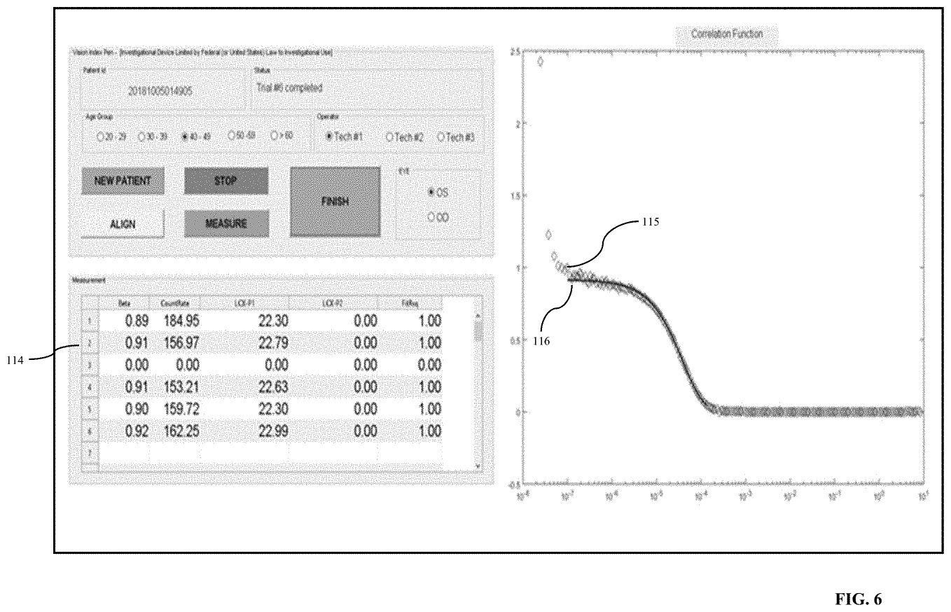

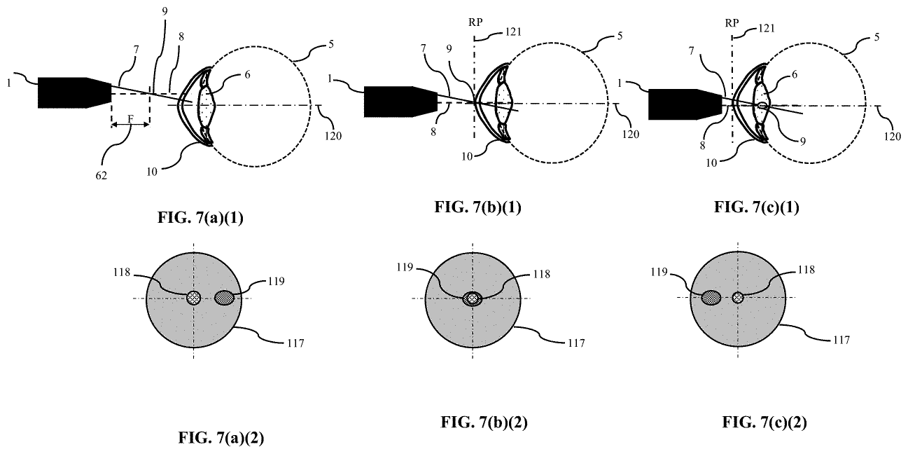

Referring to FIGS. 5 and 6, the measurement of the LCX index includes a sequence of steps. The measurement sequence is initiated at 105 by activating the START button on the interactive display (see FIG. 6). Next, as indicated at 107, the location of the measurement volume inside the eye lens 6 is set. The operator activates the ALIGNMENT button on the interactive display to initiate alignment. When the ALIGNMENT button is activated, the primary coherent source 26 in FIG. 1 is enabled in the low power mode (nominal signal power=30 μW but not to exceed 50 μW) and the secondary coherent source 29 in FIG. 1 is enabled at nominal optical power level of 30 μW, but not exceeding 50 μW. In the ALIGNMENT mode, two coherent beams emanate from the tip of the eye probe 1 in FIG. 1. The operator follows the procedure detailed below ad illustrated in FIG. 7 to locate the measurement volume 9 in the interior region of the lens 6 in FIG. 1.

Next, as indicated at 108 the patient is informed to remain still while the measurement is underway and eye probe 1 is locked into position. Thereafter, as indicated at 109, the MEASURE button on the interactive display is activated by the operator. The output signal power of the primary coherent source LD1 26 in FIG. 1 is increased to a nominal eye safe value of 70 μW (not to exceed 100 μW), and the secondary coherent source 29 in FIG. 1 is either disabled or enabled at lower power level (nominal signal power=10 μW) if the two coherent sources have different emission wavelengths. An audible beep is an indication that the measurement is in progress. Cessation of the beeping sound indicates completion of the measurement and the patient is instructed to relax. At 110, a check for validity of the correlation data is performed. The validation of data is based on two criteria:

The average backward scattered signal count rate <n> should be within a predetermined range, for example, <n> must be within the range of 25 thousand counts per second to 1 million counts per second; and

The self-beating efficiency factor β, for dynamic light scattering should be within a predetermined range, for example, β must be greater than 0.5 and less than 1.0.

If the measured correlation function fails either of the two validation tests above, the measurement can either be repeated by starting back at 105 or the measurements for the patient can be aborted at 111. If the data is determined to be valid, a real-time non-linear least squares curve-fit to the measured correlation data is performed at 112 to recover a numerical estimate of the average lens crystallin index (LCX_1) and its standard deviation (LCX_2). The entries in the running data table on the interactive display is then updated at 106 with the latest numerical estimates. Typically, three to five independent measurements are taken at each measurement volume 9 (FIG. 1) in the interior of the lens 6. At 113, the measured data for the patient is archived. All patient specific data is archived on a hard drive or similar storage device in a sub-folder identified by the time stamp at the time of measurement.

FIG. 6 shows a screen shot of the interactive display in use for a typical measurement as well as some of the buttons and display information detailed above. More specifically, the data table 114 summarizes results of a sequence of measurements. The graphical window shows a plot of the last measured correlation data 115, together with the results of the curve fit 116.

The alignment procedure, Step 2 in FIG. 5, is described with reference to FIG. 7. The eye probe 1, is typically mounted on the common eye-exam slit-lamp apparatus found in every optician facility. The eye probe attaches to the slit-lamp using the tonometer mounting round rod, requiring no modification of the slit-lamp apparatus. For precise axial positioning, the eye probe is mounted on a manual or motor driven micrometer translational stage. It is to be understood, that while FIG. 7 describes alignment along the optical axis, in general, a plurality of micrometers can provide positioning in 3-D space.

As illustrated in FIGS. 7(a)(1)-7(c)(2), the alignment procedure involves three steps. Referring to FIGS. 7(a)(1) & 7(a)(2), the measurement volume 9 is first positioned in front of the anterior surface 10 of the cornea. The frontal view 117 of the anterior surface 10 of the cornea shows two spots 118 and 119. The circular spot 118 is formed by beam 8 as it enters the anterior surface 10 of the cornea and travels into the eye 5. The first step of the alignment procedure is the alignment of the optical axis of the aligned position, that is, the optical axis 120 of the eye 5 and the optical axis 8 of the eye probe 1. At the aligned position these two optical axes 8 and 120 are congruent, and there are no other visible reflections from beam 8. The elliptical spot 119 is formed by beam 7 as it enters the anterior surface 10 of the cornea and travels into the eye 5. The two spots are easily distinguishable. Contrast between the spots can be enhanced further if different emission wavelengths are used for the two coherent sources 26 and 29 in FIG. 1. Once the congruency of the optical axis 8 of the eye probe 1 and the optical axis 120 of the eye 5 is established, the eye probe 1 is translated, using a micrometer (not shown), toward the anterior surface of the cornea 10. The elliptical spot 119 is seen to move from right to left until it merges with the circular spot 118 as illustrated in FIGS. 7(b)(1) & 7(b)(2). The merging of the two spots at the anterior surface of the cornea 10 defines the point of intersection 121 of the beams 7 and 8 on the optical axis. A plane perpendicular to the optical axis and containing the intersection point 120 serves as the axial reference plane (RP) 121 for precise axial positioning of the measurement volume 9 in FIGS. 7(c)(1) & 7(c)(2). At this axial location FIGS. 7(b)(1) & 7(b)(2), the digital micrometer (not shown) is reset to zero, as a reference for axial depth indication. The axial measurement volume 9 is precisely located by rotating the micrometer to the desired axial location as illustrated in FIGS. 7(c)(1) & 7(c)(2). For example, axial movement of 5 mm will typically place the measurement volume 9 in the nuclear region of the lens 6. In this axial position, the circular spot 118 also serves as fixation target for the patient during measurement.

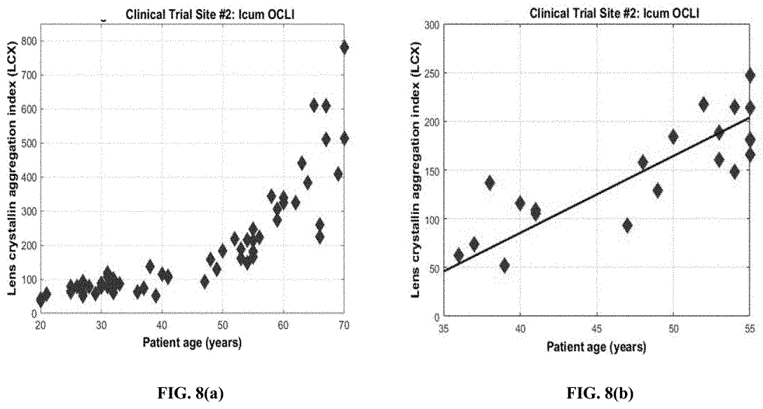

The utility of the VIPDx system was established through an observational clinical trial, the results of which are summarized in FIG. 8(a), which shows the utility of the Lens Crystallin Index (LCX) to quantitate the aggregation state of the protein molecules in the eye lens cytoplasm as a function of age. The expanded view, FIG. 8b, shows that the LCX value changes significantly for human subjects in the age range of 35 years to 55 years. This age window is critical for administration of preventive therapeutic agents for intercepting the progression of presbyopia and cataracts.

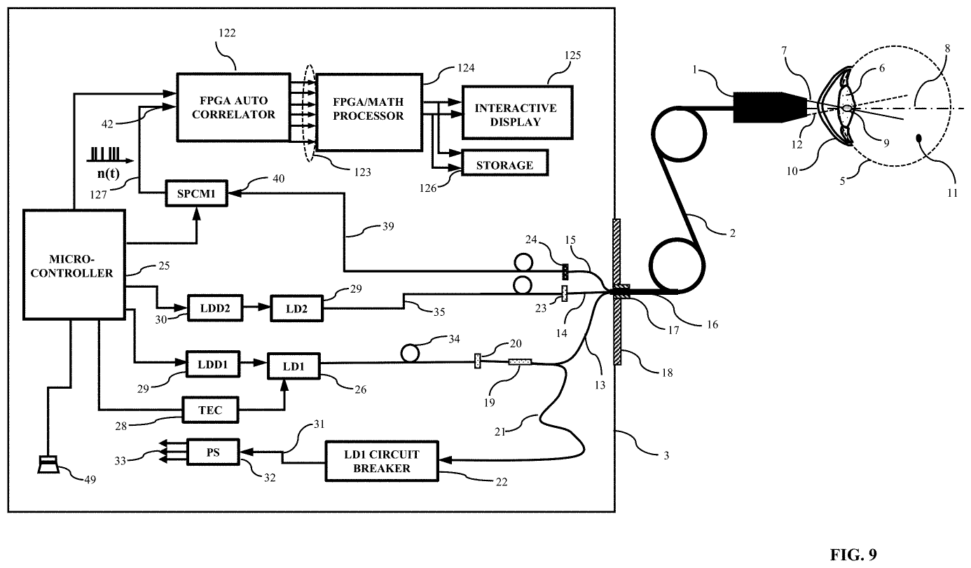

Another embodiment of the VIPDx system is illustrated in FIG. 9, which shows an implementation using a hardware/firmware solution based on a field programmable gate array (FPGA). The computer and correlator of the system of FIG. 1, are replaced by a FPGA implementation of an autocorrelator 122. At the end of the 10-s measurement, (see FIG. 5), the data vector 123 of the second order intensity autocorrelation, together with total samples and total counts is passed to the FPGA-based math processing unit 124. Detailed schematics for the FPGA autocorrelator 122 and the FPGA Math Processor 124 are discussed below. LCX values are extracted from the correlation data and displayed on the interactive display 125 and archived in a storage device 126. It is understood that the storage device 126 can be localized within the enclosure of the electronics module 3 or externally disposed and connected thereto by suitable means.

Dynamic light scattering from weakly scattering systems requires the use of single photon detection followed by a digital correlator, as described in FIG. 1; however, these digital correlators are prohibitively costly and usually available only as standalone units. Broad community use of an in vivo VIPDx ophthalmic diagnostic system requires a low cost digital correlator which is integrated into the system as illustrated in FIG. 9. The output n(t) of a single photon counting module 40 is an asynchronous pulse train, each pulse having a nominal width between 10 ns and 50 ns, and having an instantaneous pulse rate which is proportional to the intensity of the light incident on the photodetector. The autocorrelation of the pulse train n(t) is expressed as summation of the product of the n(t) and its delayed version n(t+τ). For a time-sampled system with a sampling time Ts and average count rate Φ counts per second, the average number of counts <n> for each sample period is <n>=Φ Ts. The number of counts per sample period will be proportional to the randomly varying scattered intensity corresponding to the movement of the scatterers. The autocorrelation function of the asynchronous pulse train, at discrete delay time increments is,

$${G}_{m} = {\sum }_{p = 1}^{{N}_{s}} {n}_{p} {n}_{p + m} \qquad ( Equation 1 )$$

where nₚ is the number of pulses in the p^{th} sample interval and m is the m^{th} delay time increment corresponding to τₘ, Nₛ corresponds to the total number of sample intervals during the measurement. Unbiased inversion of the correlation function requires an unbiased estimate of the true baseline, which refers to the uncorrelated value defined at infinite delay time. One estimate referred to as the measured baseline allocates a group of delay channels at large delays compared with the sampling time. A calculated base line is estimated from the average count rate per sampling interval. Herein, the baseline is estimated using a running counter for total counts, that is,

$${G}_{∞} = {( {\sum }_{p = 1}^{{N}_{s}} {n}_{p} )}^{2} \qquad ( Equation 2 )$$

The second order correlation function G(τ) is related to the first order correlation function g(τ), through the Siegert relation,

G(τ)=G_{∞}[1+β|g(τ)|²] (Equation 3)

where β is the self-beating efficiency factor, typically, in the range 0.5 to 1.0, and G_{∞}, is the baseline. Some data inversion techniques require the recovery of the first order correlation function while others use the second order function as it is measured. The physical information of the scattering system is embedded in the definition of the first order correlator function. As an example, the first order electric field correlation function is related to a distribution G(F), representing the weighted contribution of the characteristic decay rates F, through an integral relationship,

β^{1/2}|g(τ)=∫ΓₘᵢₙΓₘₐₓG(Γ)e^{-Γτ}dΓ (Equation 4)

Derivation of equation (4) assumes an independent scattering system, with scatterers executing Brownian motion. The former assumption applies to low concentration systems, typically below 0.01% by weight. It should be noted that the lens cytoplasm has a protein concentration exceeding 35% by weight. Further, for Brownian motion F is a function of the scattering angle, refractive index and viscosity of the medium hosting the moving scatterers, the effective hydrodynamic radius of the scatterers, and wavelength of the coherent source. In general, large values of F correspond to fast moving components while small values correspond to slow moving components. Several methods are available for inverting the integral equation (4) for extracting G(F), and from that the distribution of hydrodynamic radius, or distribution of molecular weights.

Data inversion of the in vivo measurements from the lens cytoplasm has proven to be very challenging due to the high concentration of proteins in the lens as well as the degradation in the quality of the correlation function due to involuntary movement of the patient, such as blinking. Thus, it is prudent and necessary to extract as few parameters as possible from the measured correlation data. For this reason, a method based on cumulants expansion of the basis function in equation (4) is preferred for inverting measured correlation data. A linear approximation to equation (4) is,

$$b = ln [ {β}^{\frac{1}{2}} \vert {g}^{( 1 )} ( τ ) \vert ] = 0.5 ln ( β ) - \overline{Γ} τ + \frac{{κ}_{2}}{2 !} {τ}^{2} - \frac{{κ}_{3}}{3 !} {τ}^{3} … \qquad ( Equation 5 )$$

where κ_{I} defined as the i^{th} cumulant, and Γ̅ is the average value of the linewidth. The discrete form of equation (5) becomes

$${b}_{m} = \sqrt{( \frac{{G}_{m}}{{G}_{∞}} - 1 )} \qquad ( Equation 6 )$$

The mean square error between the model, equation (5) and the normalized data equation (6), is given by,

$${ɛ}^{2} = {\sum }_{m = 1}^{M} {[ {b}_{m} - ( {P}_{1} - {P}_{2} τ + \frac{1}{2} {P}_{3} {τ}^{2} ) ]}^{2} \qquad ( Equation 7 )$$

where Pᵢ are the unknown parameters to be determined from the data. The minimization constraint of equation (7) leads to a set of linear equations which can be solved to obtain the best estimates of Pᵢₛ. It is common practice to use software algorithms to directly solve equation (7). However, the present invention describes a hardware solution which can be implemented using field programmable gate arrays (FPGA). The following equations are developed to extract P₁, P₂, and P₃.

$$\begin{matrix}{P}_{1} = \frac{{q}_{1} ( {S}_{3}^{2} - {S}_{2} {S}_{4} ) + {q}_{2} ( {S}_{1} {S}_{4} - {S}_{2} {S}_{3} ) + {q}_{3} ( {S}_{2}^{2} - {S}_{1} {S}_{3} )}{{S}_{2}^{2} - 2 {S}_{1} {S}_{2} {S}_{3} + {MS}_{3}^{2} + {S}_{1}^{2} {S}_{4} - {MS}_{2} {S}_{4}} & ( Equation 8 ) \\ {P}_{2} = \frac{\begin{matrix}{q}_{1} ( {S}_{2} {S}_{3} - {S}_{2} {S}_{4} ) + \\ {q}_{2} ( {MS}_{4} - {S}_{2}^{2} ) + {q}_{3} ( {S}_{1} {S}_{2} - {MS}_{3} )\end{matrix}}{{S}_{2}^{3} - 2 {S}_{1} {S}_{2} {S}_{3} + {MS}_{3}^{2} + {S}_{1}^{2} {S}_{4} - {MS}_{2} {S}_{4}} & ( Equation 9 ) \\ {P}_{3} = \frac{2 [ \begin{matrix}{q}_{1} ( {S}_{2} {S}_{3} - {S}_{2} {S}_{4} ) + \\ {q}_{2} ( {MS}_{4} - {S}_{1} {S}_{2} ) + {q}_{3} ( {S}_{1}^{2} - {MS}_{3} )\end{matrix} ]}{{S}_{2}^{3} - 2 {S}_{1} {S}_{2} {S}_{3} + {MS}_{3}^{2} + {S}_{1}^{2} {S}_{4} - {MS}_{2} {S}_{4}} & ( Equation 10 )\end{matrix}$$

where M is the number of data points in the correlation function or the length of the normalized vector [bₘ], and the q_{1,2,3} and S_{1,2,3,4} are various summations given by,

$$\begin{matrix}{q}_{1} = {\sum }_{m = 1}^{M} {b}_{m} , {q}_{2} = {\sum }_{m = 1}^{M} {b}_{m} {τ}_{m} , \\ {q}_{3} = {\sum }_{m = 1}^{M} {b}_{m} {τ}_{m}^{2} & ( Equation 11 ) \\ {S}_{1} = {\sum }_{m = 1}^{M} {τ}_{m} , {S}_{2} = {\sum }_{m = 1}^{M} {τ}_{m}^{2} , \\ {S}_{3} = {\sum }_{m = 1}^{M} {τ}_{m}^{3} , {S}_{4} = {\sum }_{m = 1}^{M} {τ}_{m}^{4} & ( Equation 12 ) \\ β = {10}^{2 {P}_{1}} \\ \overline{Γ} = {P}_{2} \\ {σ}_{Γ} = \sqrt{{P}_{3}} & ( Equation 13 )\end{matrix}$$

where σ_{Γ} is the standard deviation of distribution G(Γ) with average value Γ̅. The lens crystallin aggregation index, LCX is inversely proportional to Γ̅. A scaling factor, based on Brownian motion, and some of the physical parameters of the system is used to convert Γ̅ to LCX. While it generally understood that the motion of the crystallins in the lens fiber cells is far from Brownian motion, none the less clinical observations shows that the extracted LCX parameter shows good correlation with age (see FIG. 8).

Another difficulty encountered with in vivo DLS measurements is the momentary increases or decreases in the instantaneous counts in a given sample (or group) bin. These abnormal events lead to distortion of the measured correlation function. None of the existing correlator designs can filter these events in real time and at the fastest sampling frequency. Some techniques have implemented exclusion of abnormal counts by first detecting these events and then enabling electronic shutters to exclude these counts from being used in the accumulation of the correlation channels. However, these mitigatory techniques are necessarily several orders of magnitude slower than the fastest sampling rates of the correlator and not very effective. The present invention provides an electronic circuitry for abnormal event detection and exclusion of the momentary events from being a part of the correlation function at the fastest sampling rate, for example 25 ns, with no latency. Operation of the circuit is described below.

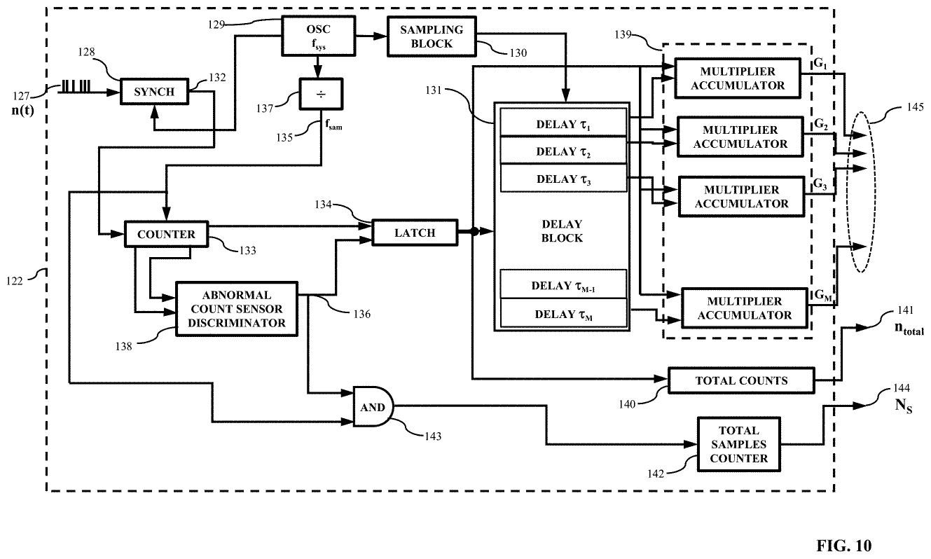

FIG. 10 is a schematic of the FPGA-based digital autocorrelator 122 of FIG. 9. Asynchronous voltage pulses 127, corresponding to the backward scattered signal, typically have a nominal width of 20 ns and count rate in the range of 100 counts per second to 10 million counts per second. A synchronizer 128 derandomizes the arrival of the pulses within the sampling time and synchronizes the said pulses with the system clock 129. For access to fast moving scatterers, such as, lens proteins, and similar small molecules, the system clock should have frequency in the range 100 MHz, to 500 MHz. The sampling block 130 generates the delay time vector [τ] 131 defining the delay time increments at which the autocorrelation function is computed. For example, a delay time increments in the range from 25 ns to 1 s, using logarithmically spaced delay increments.

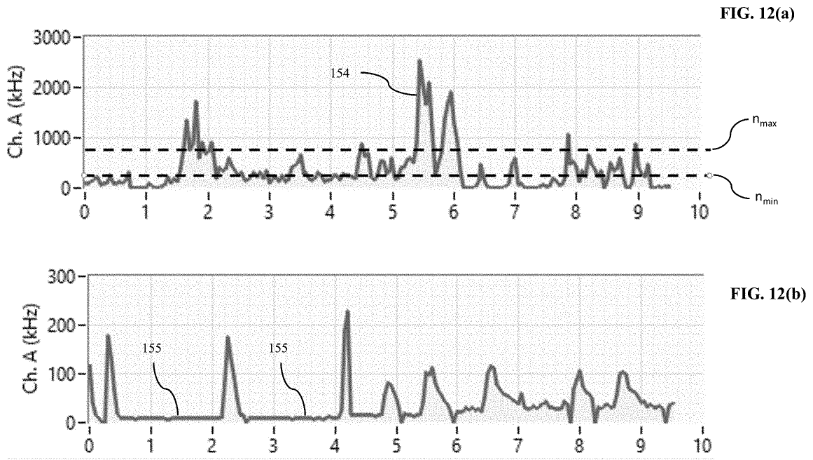

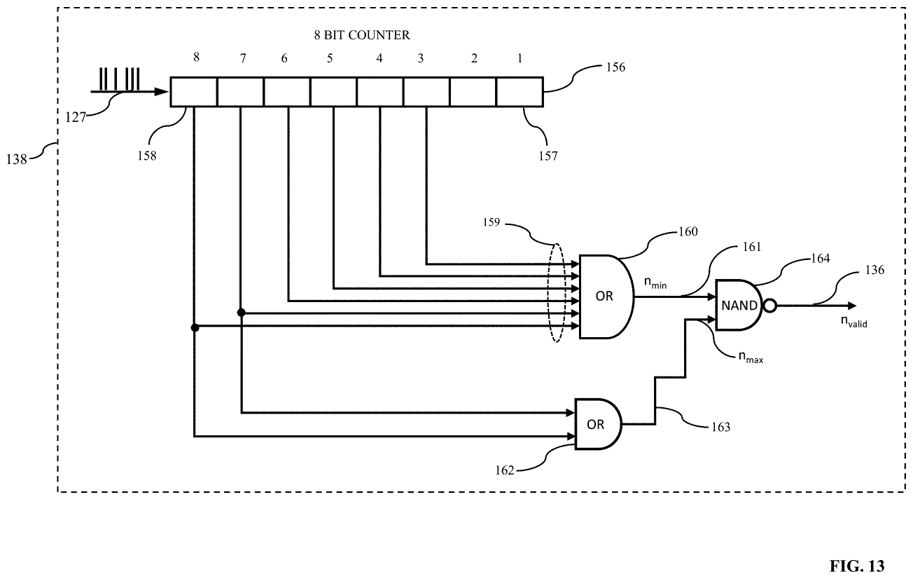

Continuing with reference to FIG. 10, the synchronized pulses 132 are fed into a counter 133, with depth determined by the fastest sampling time and the maximum count rate of the asynchronous voltage pulses 127. The counter 133 is sampled and its contents transferred to the latch 134 at the rising edge of a sampling clock 135, provided the n_{valid} flag 136 is HIGH, otherwise the counter content is overwritten during the next sampling interval. A divider 137 generates the sampling clock 135. The abnormal count sensor and discriminator 138 generates the n_{valid} flag 136 by sampling the logic states of the counter bits. Operation of the abnormal count sensor and discriminator 138 is discussed below with reference to FIG. 12 and FIG. 13. Counter value falling outside the specified range nₘ in to nₘₐₓ, during each sampling period, are rejected by the abnormal count sensor and discriminator 138, leading to a distortion free measurement of the autocorrelation of the backward scattered signal.

After an initial set-up time equal to the maximum delay increment, all delay registers in the delay block 131 contains valid counts corresponding to nm. These register values are updated at the sampling clock rate. At the rising edge of each sample clock the counts in the delay registers are shifted into the multiplier/accumulator blocks 139 which compute the correlation function, according to the definition of Equation 1 for all delay increments M. These accumulations will continue until the end of the measurement signal (not shown), is received. A separate counter 140 accumulates the total counts nₜₒₜₐₗ 141 and another counter 142, in combination with the AND gate 143 accumulates the total number of valid samples Nₛ 144 used in the accumulation of the autocorrelation vector [G] 145.

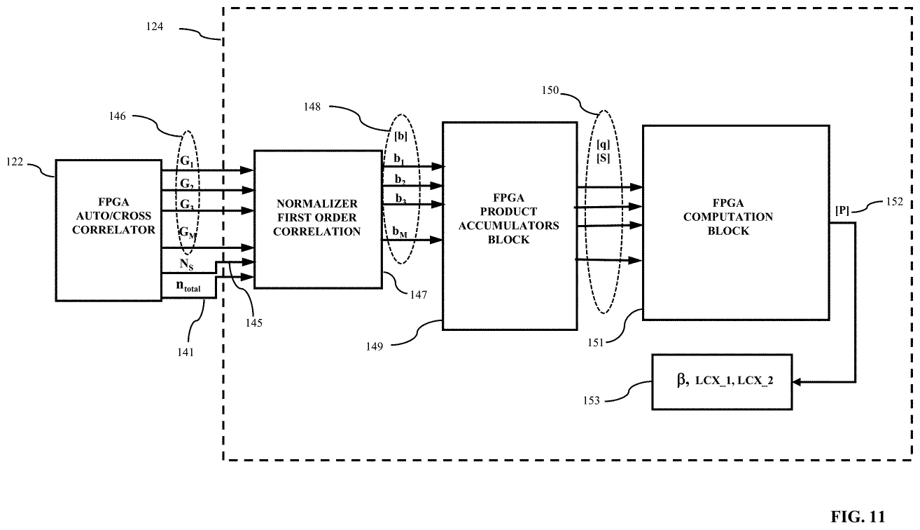

Several data inversion methods for extracting physical information from the scattering system are in use. Invariably, all data inversion algorithms run on computers to extract the relevant information from the measured second order autocorrelation function. There exists a need for autonomous DLS systems with integrated hardware for direct inversion of the measured autocorrelation function. FIG. 11 shows a hardware implementation of a modified method for extracting the lens crystallin index (LCX) from the measured autocorrelation function described by Equation 1. The data extraction procedure requires evaluation of products and summations as indicated in Equation 11 and Equation 12. The un-normalized correlation data vector [G] 146, total counts mow 141 and total samples Nₛ 144 are processed by the data normalization block 147 to generate the normalized first order electric field autocorrelation vector [b] 148. The FPGA products and summations block 149 evaluates the various summation needed, given in Equation 11 and Equation 12. The intermediate computation data vectors [q] [S] 150 are passed to the FPGA computation block 151 which computes output parameter vector [P] 152 by evaluating Equation 9 and Equation 10. One of the vector components of [P] is the mean value of the lens crystallin index (LCX_1) 153. The [P] vector is archived to the storage device 126 of FIG. 9, and the parameters β, LCX_1 and LCX_2 values are displayed on the interactive display 125 of FIG. 9.

The embodiment illustrated in FIG. 10 shows the use of an abnormal event count sensor and discriminator 138 to reject counter values outside the specified range. Involuntary eye blinking and reflex can cause abrupt changes in the counter value, leading to distortion of the measured autocorrelation. FIGS. 12(a) & 12(b) illustrates this problem, wherein FIG. 12(a) shows the abrupt increases 154 in the counts due to reflex, while FIG. 12(b) shows the obscuration 155 of the signal due to blinking. These abnormal count events are disruptive and produce distortions in the measured autocorrelation function of the scattered signal.

With reference to FIG. 13, the operation of the abnormal count sensor and discriminator 138 is herein described below. The abnormal count sensor and discriminator 138 produces a valid count flag 136, when the counter value is within the specified range nₘᵢₙ to nₘₐₓ. The lower bound rejects noise pulses which dominate when the back-scattered signal is obscured from entering the detection optics 12 in FIG. 1, for example, due to involuntary eye blinking as discussed above. The upper bound defines the maximum allowable counts, thereby rejecting momentary increases in the signal count due specular reflection (reflex) entering the detection optics 12 in FIG. 1, also arising from involuntary eye movement. In the illustrative schematic of FIG. 13, an 8-bit counter 156 is used to count the number of pulses 127 detected during each sampling interval. Bit-1 157 is the least significant bit (LSB) and bit-8 158 is the most significant bit (MSB). The logic state of bits 3 to 8 159 is detected by a multiple input OR gate 160. When any of the bits between bit 3 and bit 8 is in a logic HIGH state, the output 161 is HIGH, indicating that the count exceeds the minimum value nₘᵢₙ, which in the illustrated circuit of FIG. 13 corresponds to a count value of 3. Logic state of bits 7 and 8 is sensed by a multiple input OR gate 162, which generates a logic HIGH at the output 163 if either of bit 7 or bit 8 is HIGH, indicating that the counter value exceeds the maximum allowable value nₘₐₓ. In the circuit illustrated in FIG. 13, inputs 161 and 163, to the NAND gate 164 are in logic state LOW, when the counter value is in the range of 4 to 127, generating a HIGH at the output 136 of the NAND gate 164. Subsequently, the counter value is moved into the latch 129 of FIG. 10. When the counter value is greater than nₘₐₓ and less than nₘᵢₙ, the n_{valid} signal 136 is LOW and the counter value is overwritten at the next sample clock edge and not moved to the latch 134.

The discrimination of abnormal counts takes place in real-time and at the fastest sampling interval. While the operation of the said the abnormal count sensor and discriminator has been described with reference to in vivo measurements from a human eye, it is appreciated that abnormal count sensor and discriminator 138 can also be used in other situations, for example, discrimination against deleterious signal due to the presence of large contaminant particles in a dilute suspension of small molecules. It should also be appreciated that the operation of the abnormal count sensor and discriminator 138 is independent of the counter depth, and an 8-bit counter is merely used to simplify the discussion herein.

Although illustrative embodiments of the present invention have been described herein with references to the accompanying drawings, it is to be understood that the invention is not limited to those precise embodiments, and that various other changes and modifications may be affected herein by one skilled in the art without departing from the scope or spirit of the invention.

What is claimed is:

1. A system for dynamic light scattering measurement, comprising:
b. first and second light sources;
c. first and second gradient index lenses;
d. first and second optical paths from the first and second light sources to an eye by way of the first and second gradient index lenses, respectively;
e. an optical splitter configured to split light, originating from the first and second light sources and scattered from the eye, into first and second light signals;
f. first and second light detectors configured to detect the first and second light signals, respectively; and
g. a correlator configured to correlate respective first and second signals output from the first and second light detectors.

2. The system according to claim 1, wherein the first and second light sources are configured to produce different wavelengths of light.

3. The system according to claim 1, wherein the correlator is a cross-correlator.

4. The system according to claim 1, wherein the correlator is an FPGA autocorrelator.

5. The system according to claim 1, wherein the correlator includes an abnormal count sensor and discriminator configured to reject counter values falling outside a specified range to inhibit distortion of measurement.

6. The system according to claim 1, further comprising first and second single-mode optical fibers configured to define a portion of the first and second optical paths, respectively.

7. The system according to claim 6, wherein the first and second single-mode optical fibers and the respective first and second gradient lenses are incorporated into respective first and second transreceivers extending through a body of an eye probe.

8. The system according to claim 1, further comprising a circuit breaker configured to shut down at least the first light source if emitted optical power from the first light source reaches a threshold.

9. A method for extracting a lens crystallin index, comprising:
h. obtaining measurements of backscattered light originating from a plurality of light sources and reflected by an eye;
i. performing a data inversion of at least some of the obtained measurements to obtain inverted data;
j. performing a fitting of at least some of the inverted data; and
k. generating a lens crystallin index data plot based on a result of the fitting.

10. The method according to claim 9, wherein obtaining measurements further includes:
l. splitting the backscattered light into a plurality of light signals;
m. detecting the plurality of light signals; and
n. correlating the plurality of light signals to determine a counter value, wherein the obtained measurements are counter values.

11. The method according to claim 9, wherein at least some of the obtained measurements are discarded and not used in the data inversion.

12. The method according to claim 11, wherein the obtained measurements within a counter value range are used in the data inversion and wherein the obtained measurements outside of the counter value range are not used in the data inversion.

13. The method according to claim 9, wherein at least some of the inverted data is not used in the fitting.

14. The method according to claim 13, wherein the inverted data at or below an upper limit is used in the fitting and the inverted data above the upper limit is not used in the fitting.

15. A non-transitory computer readable medium having stored thereon instructions which, when executed by one or more processors, cause the one or more processors to:
o. obtain measurements of backscattered light originating from a plurality of light sources and reflected by an eye;
p. perform a data inversion of at least some of the obtained measurements to obtain inverted data;
q. perform a fitting of at least some of the inverted data; and
r. generate a lens crystallin index data plot based on a result of the fitting.

16. The medium according to claim 15, wherein at least some of the obtained measurements are discarded and not used in the data inversion.

17. The medium according to claim 16, wherein the obtained measurements within a counter value range are used in the data inversion and wherein the obtained measurements outside of the counter value range are not used in the data inversion.

18. The medium according to claim 15, wherein at least some of the inverted data is not used in the fitting.

19. The method according to claim 18, wherein the inverted data at or below an upper limit is used in the fitting and the inverted data above the upper limit is not used in the fitting.
